(19) United States
(12) Plant Patent Application Publication
(10) Pub. No.: US 2017/0127585 P1
(43) Pub. Date: May 4, 2017

(54) Citrus reticulata blanco plant named 'Tamnaneunbong'

(50) ;

(71) Applicant: THE RURAL DEVELOPMENT ADMINISTRATION, REPUBLIC OF KOREA, Chungcheongnam-Do (KR)

(72) Inventors: Jae-Ho Park, Seogwipo-Si (KR); Su-Hyun Yun, Jeollabuk-Do (KR); Dong-Hoon Lee, Gyeonggi-Do (KR); Sang-Woog Koh, Jeju-Do (KR); Hyun-Joo An, Seogwipo-Si (KR); I-Ung Yang, Seogwipo-Si (KR)

(21) Appl. No.: 14/756,932

(22) Filed: Oct. 30, 2015

(51) Int. Cl. A01H 5/08

(52) U.S. Cl. PLT/201

(57) ABSTRACT

A new and distinct Citrus Reticulate Blanco plant named “Tamnaneunbong”, characterized by its upright plant type and strong vigor, short oval ovary shape, germination stage in middle of April, blooming stage in late May, fruits start coloring in late October, mature stage in late March, acidity of the fruit juice at about 1.51%, leaf length at about 10.1 cm and leaf width at about 4.3 cm, and length of leaf petiole at about 3.6 cm.

Latin name of the genus and species of the plant claimed: Citrus reticulata Blanco.

Variety denomination: ‘Tamnaneunbong’.

### BACKGROUND OF THE INVENTION

The new and distinct cultivar is a product of a breeding program by the inventors. The ovules developing of ‘Shiranui’ was carried out in 2000. The characteristic investigation of these nucellar seedlings was done from 2007 to 2010 in a greenhouse. The first screening was carried out in 2007, and the last screening was carried out in 2010. One new variety was picked and named ‘Tamnaneunbong’.

After hybridization of the parents, the variety was planted by asexual propagation for three generations and there was no genetic variation, therefore, its traits are stable.

The fruit characteristics of Tamnaneunbong are in the table below. The test carried out from 2007 to 2010, in Citrus Research Station. Shiranui was 5 years old in 2007. The Tamnaneunbong was the nucellar seedling in this table.

| | | Fruit | | Fruit | Peak | Thickness | Seed | | | Sugar |
|---|---|---|---|---|---|---|---|---|---|---|
| Survey | | weight | Flesh | shape | height | of the skin | grain | Sugar | Acidity | acid |
| date(yy.mm.dd) | Variety | (g) | rate(%) | index | (mm) | (mm) | number | (° Bx) | (%) | ratio |
| 07.1.24 | Tamnaneunbong | 268.4 | 77.5 | 94 | 15.6 | 2.9 | 0 | 14.4 | 1.28 | 11.24 |
| | Shiranui | 315.2 | 69.4 | 100 | 13.8 | 5.3 | 0 | 12.4 | 1.14 | 10.92 |
| 08.2.14 | Tamnaneunbong | 254.8 | 75.7 | 89 | 12.8 | 3.7 | 0 | 13.6 | 1.44 | 9.43 |
| | Shiranui | 172.4 | 72.4 | 98 | 4.5 | 4.3 | 0 | 12.8 | 1.32 | 9.71 |
| 09.2.17 | Tamnaneunbong | 364.8 | 72.6 | 93 | 17.0 | 4.2 | 0 | 15.3 | 1.28 | 11.97 |
| | Shiranui | 322.4 | 75.6 | 92 | 15.1 | 4.1 | 0 | 13.7 | 1.14 | 11.98 |
| 10.2.24 | Tamnaneunbong | 240.3 | — | — | — | — | 0 | 15.3 | 1.46 | 10.47 |
| | Shiranui | 157.0 | — | — | — | — | 0 | 12.2 | 1.46 | 8.35 |

Fruit shape index=The transverse diameter÷The longitudinal diameter×100

Additional fruit characteristics of Tamnaneunbong are in the table below. Shiranui was 8 years old in 2010. The Tamnaneunbong was grafted onto five years old Shiranui three years ago.

| | | Fruit | | Fruit | Peak | Thickness | Seed | | | Sugar |
|---|---|---|---|---|---|---|---|---|---|---|
| Survey | | weight | Flesh | shape | height | of the skin | grain | Sugar | Acidity | acid |
| date(yy.mm.dd) | Variety | (g) | rate(%) | index | (mm) | (mm) | number | (° Bx) | (%) | ratio |
| 10.1.27 | Tamnaneunbong | 280.9 | 77.7 | 95 | 10.7 | 3.6 | 0 | 15.3 | 1.45 | 10.67 |
| | Shiranui | 255.6 | 77.0 | 97 | 9.2 | 3.8 | 0 | 14.5 | 1.20 | 12.08 |
| 10.3.4 | Tamnaneunbong | 237.7 | 76.5 | 93 | — | 3.8 | 0 | 17.5 | 1.51 | 11.74 |
| | Shiranui | 258.8 | 73.0 | 93 | — | 4.1 | 0 | 16.0 | 1.22 | 13.17 |

### SUMMARY OF THE INVENTION

The cultivar ‘Tamnaneunbong’ has not been observed under every possible environmental conditions. Therefore, the may be variations in the phenotype due to varying environmental conditions such as temperature, day length and light intensity. However, the genotype will not vary.

The following traits have been repeatedly observed and are determined to be the characteristics of the new cultivar of Citrus reticulata Blanco. These attributes in combination distinguish ‘Tamnaneunbong’ as unique from and all other varieties of Citrus reticulata Blanco known to Inventors.

1. The plant type of ‘Tamnaneunbong’ is upright and the tree vigor is strong.

2. Ovary shape of ‘Tamnaneunbong’ is short oval.

3. The fruit color is orange.

4. Germination stage is in the middle of April.

5. Blooming stage is in late May.

6. Fruits start coloring in late October.

7. The mature stage is in late March.

8. Acidity of the fruit juice is about 1.51%.

9. The leaf length is about 10.1 cm and the leaf width is about 4.3 cm.

10. The length of leaf petiole is about 3.6 cm.

Plants of the new cultivar ‘Tamnaneunbong’ are similar to plants of the seed parent ‘Shiranui’ in most horticultural characteristics, however, the plants of the new cultivar ‘Tamnaneunbong’ produce fruit that are more acidic than the parent. The fruit produced by ‘Tamnaneunbong’ start coloring in late October and mature in late March.

The new cultivar differs from the closest comparable plants as shown in the tables below:

TABLE 1

| Denom- | | | |
|---|---|---|---|
| ination | Characteristic in | | |
| of similar | which the similar | The expression of the characteristic | |
| variety | variety is different | Similar variety | Candidate variety |
| Shiranui | Plant: type | medium | upright |
| | Plant: tree vigor | medium | strong |
| | Ovary: shape | depressed globose | short oval |
| | Fruit juice: acidity | 1.22% | 1.51% |
| | Leaf: leaf length | 8.0 cm | 10.1 cm |
| | Leaf: leaf width | 3.0 cm | 4.3 cm |
| | Leaf petiole: length | 4.1 mm | 3.6 mm |
| | Fruits start coloring | in early October | in late October |
| | Mature stage | in early March | in late March |

TABLE 2

| | | | | | | | | | | | Application | | Similar | |
|---|---|---|---|---|---|---|---|---|---|---|---|---|---|---|
| | | | | | | | | | | | variety: | | variety: | |
| | Charac- | Forms of Expression | | | | | | | | | Tamnaneunbong | | Shiranui | |
| NO | teristics | 1 | 2 | 3 | 4 | 5 | 6 | 7 | 8 | 9 | No | Data | No | Data |
| 1 | Plant: Type | upright | | | | medium | | | | pendu- | 1 | | 5 | |
| | | | | | | | | | | lous | | | | |
| 2 | Yong shoot: | thin | | | | medium | | | | thick | 5 | 2.6 mm | 1 | 1.9 mm |
| | thickness | | | | | | | | | | | | | |
| 3 | Yong shoot: | very | | short | | medium | | long | | very | 7 | 20.0 cm | 7 | 15.9 cm |
| | length | short | | | | | | | | long | | | | |
| 4 | Stem: | very | | short | | medium | | long | | very | 5 | 1.9 cm | 5 | 1.5 cm |
| | internode | short | | | | | | | | long | | | | |
| | length | | | | | | | | | | | | | |
| 5 | leaf: color | light | | | | green | | | | dark | 5 | | 5 | |
| | | green | | | | | | | | green | | | | |
| 6 | leaf: length | very | | short | | medium | | long | | very | 7 | 10.1 cm | 5 | 8.0 cm |
| | | short | | | | | | | | long | | | | |
| 7 | leaf: width | very | | narrow | | medium | | broad | | very | 5 | 4.3 cm | 3 | 3.2 cm |
| | | narrow | | | | | | | | broad | | | | |
| 8 | leaf: | small | | | | medium | | | | large | 7 | 2.4 | 7 | 2.5 |
| | length/width | | | | | | | | | | | | | |
| | ratio | | | | | | | | | | | | | |
| 9 | leaf: | thin | | | | medium | | | | thick | 5 | 0.032 mm | 5 | 0.031 mm |
| | thickness | | | | | | | | | | | | | |
| 10 | Leaf petiole: | very | | short | | medium | | long | | very | 3 | 3.6 mm | 5 | 4.1 mm |
| | length | short | | | | | | | | long | | | | |
| 11 | Leaf petiole: | thin | | | | medium | | | | thick | 5 | 1.9 mm | 5 | 1.7 mm |
| | thickness | | | | | | | | | | | | | |
| 12 | Flower: petal | very | | short | | medium | | long | | very | 5 | 16.3 mm | 5 | 15.0 mm |
| | length | short | | | | | | | | long | | | | |
| 13 | Flower: petal | very | | narrow | | medium | | broad | | very | 7 | 8.1 mm | 7 | 6.7 mm |
| | width | narrow | | | | | | | | broad | | | | |
| 14 | Ovary: shape | Long | globose | depressed | applanate | cylinder | short | gourd | | | 6 | | 3 | |
| | | oval | | globose | | | oval | | | | | | | |
| 15 | Flower: | absent | | | | | | | | present | 1 | | 1 | |
| | activity | | | | | | | | | | | | | |
| | pollen | | | | | | | | | | | | | |
| 16 | Fruit: | small | | | | medium | | | | large | 3 | 0.95 | 3 | 0.95 |
| | length/width | | | | | | | | | | | | | |
| | ratio | | | | | | | | | | | | | |
| 17 | Fruit: surface | green | yellowish | light | medium | yellowish | medium | dark | red with | red | 4 | | 4 | |
| | color | | green | yellow | yellow | orange | orange | orange | orange | | | | | |
| 18 | Fruit: surface | smooth | | | | | | | | rough | 9 | | 9 | |
| | roughness | | | | | | | | | | | | | |
| 19 | Fruit: fresh | with | light | light | medium | light | medium | dark | red | purple | 5 | | 5 | |
| | color | white | green | yellow | yellow | orange | orange | orang | | | | | | |
| 20 | Fruit: skin | thin | | | | medium | | | | thick | 5 | 3.7 mm | 5 | 3.9 mm |
| | thickness | | | | | | | | | | | | | |
| 21 | Fruit juice: | low | | | | medium | | | | high | 5 | 17.5° Bx | 5 | 16.0° Bx |
| | total sol- | | | | | | | | | | | | | |
| | uble solids | | | | | | | | | | | | | |
| 22 | Fruit juice: | low | | | | medium | | | | high | 7 | 1.51% | 5 | 1.22% |
| | acidity | | | | | | | | | | | | | |
| 23 | Mature stage | very | | early | | medium | | late | | very | 7 | | 7 | |
| | | early | | | | | | | | late | | | | |
| 24 | Partheno- | absent | | | | | | | | present | 9 | | 9 | |
| | carpy | | | | | | | | | | | | | |
| 25 | Self- | absent | | | | | | | | present | 9 | | 9 | |
| | incompat- | | | | | | | | | | | | | |
| | ibility | | | | | | | | | | | | | |
| 26 | Tree vigor | weak | | | | medium | | | | strong | 9 | | 5 | 30 |

### BRIEF DESCRIPTION OF THE DRAWINGS

The accompanying colored photographs illustrate the overall appearance and distinct characteristics of the new Citrus reticulata Blanco ‘Tamnaneunbong’ showing colors as true as is reasonably possible with colored reproduction of this type. These photos were taken in 2010 in Korea. Colors in the photographs may differ slightly from the color values cited in the detailed botanical description which accurately describes the color of the ‘Tamnaneunbong’.

FIG. 1 provides multiple views of the ovaries of the fruits harvested from ‘Tamnaneunbong’ and ‘Shiranui’. The four ovaries on the left of the photograph are ovaries of the fruit harvested from ‘Shiranui’ while the four ovaries on the right of the photograph are ovaries of the fruit harvested from ‘Tamnaneunbong’.

FIG. 2 provides multiple views of typical fruits harvested from ‘Tamnaneunbong’. Top, bottom and side views of the whole, unpeeled fruit is shown as well as two cross-sectional views of the inside of the fruit.

FIG. 3 provides multiple views of typical fruits harvested from ‘Shiranui’. Top, bottom and side views of the whole, unpeeled fruit is shown as well as two cross-sectional views of the inside of the fruit

FIG. 4 illustrates in full color typical foliage and fruits of ‘Tamnaneunbong’ on the tree.

FIG. 5 illustrates in full color typical foliage and fruits of ‘Shiranui’ on the tree.

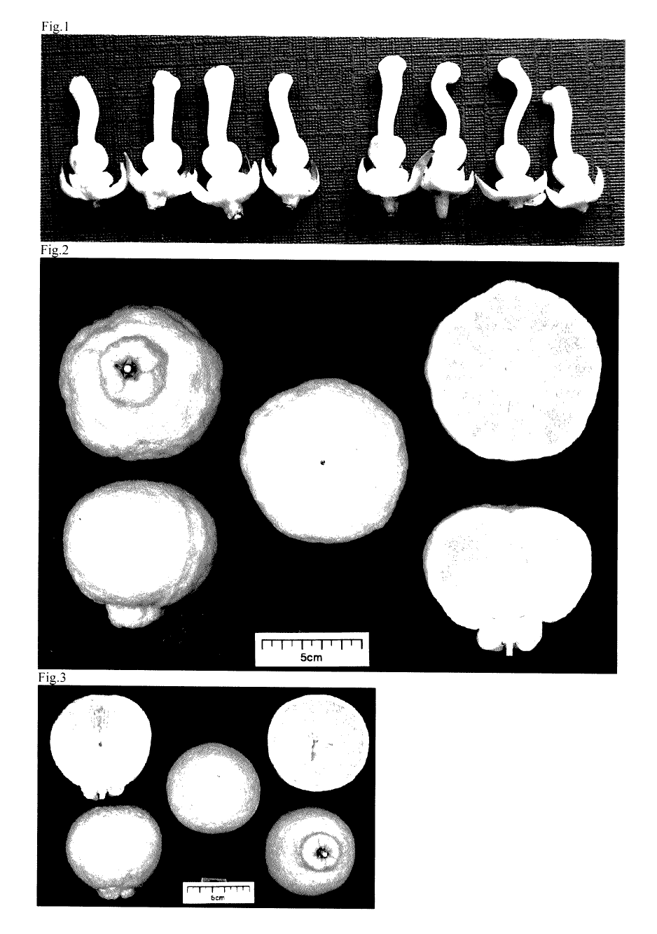

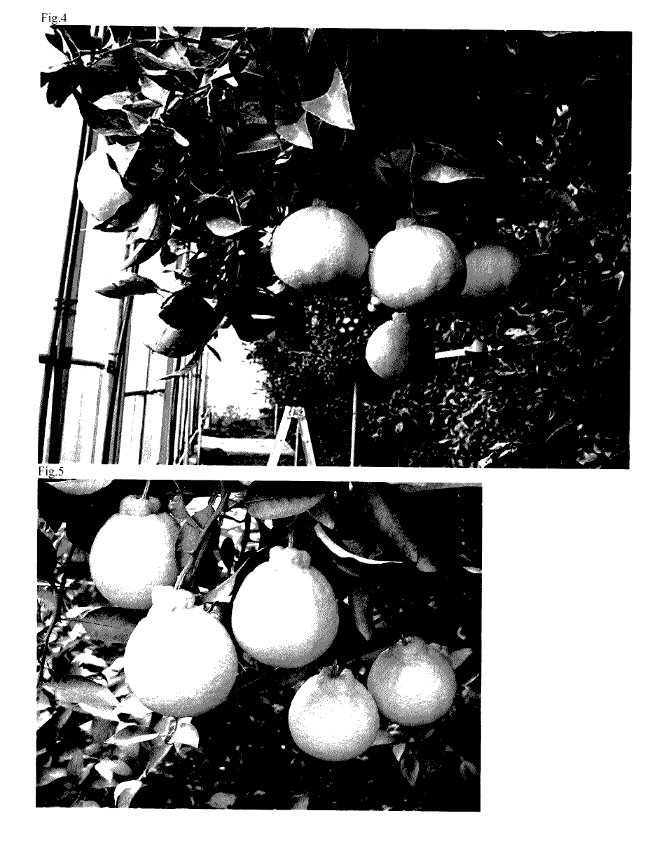

### DETAILED BOTANICAL DESCRIPTION

In the following description of the new cultivar ‘Tamnaneunbong’ as taken from tests carried from 2007 to 2010, Citrus Research Station. The following observations and measurements describe ‘Tamnaneunbong’ plants grown in Korea.
Propagation: “Tamnaneunbong’ is typically grafted onto rootstock of ‘Shiranui’
Plant:
   Age of plant described.—Approximately 3 years old.
   Vigor.—Strong.
   Plant spread.—Tree is upright.
Foliage:
   Yong shoot thickness.—2.6 mm.
   Yong shoot length.—20.0 cm.
   Stem internode length.—1.9 cm.
   Leaf color.—Green.
   Leaf length.—10.1 cm.
   Leaf width.—4.3 cm.
   Leaf length/width ratio.—2.4.
   Leaf thickness.—0.032 mm.
   Leaf petiole length.—3.6 mm.
   Leaf petiole thickness.—1.9 mm.
Flower:
   Flower petal length.—16.3 mm.
   Flower petal width.—8.1 mm.
   Ovary shape.—Short oval.
   Flower activity pollen.—Absent.
Fruit:
   Fruit shape index.—89 to 95.
   Fruit length/width ratio.—0.95.
   Fruit weight.—237 g to 365 g.
   Fruit surface color.—Medium yellow.
   Fruit surface roughness.—Rough.
   Fruit fresh color.—Light orange.
   Fruit flesh rate.—72.6% to 77.7%.
   Fruit skin thickness.—3.7 mm.
   Fruit juice total soluble solids.—17.5° Bx.
   Fruit juice acidity.—1.51%.
   Seed grain number.—0.
   Mature stage.—Late.
   Parthenocarpy.—Present.
   Self-incompatibility.—Present.
   Tree vigor.—Strong.
   Other fruit features.—For the very mature stage, ‘Tamnaneunbong’ should be protected cultured. Pruning on ‘Tamnaneunbong’ is the same as ‘Shiranui’.

What is claimed is:

1. A new and distinct cultivar of Citrus reticulata Blanco plant named ‘Tamnaneunbong’ as herein illustrated and described.
